(19) United States
(12) Patent Application Publication
(10) Pub. No.: US 2020/0245937 A1
(43) Pub. Date: Aug. 6, 2020

(54) SYSTEMS AND METHODS TO DETERMINE CENTER OF PRESSURE

(71) Applicant: The Trustees of Indiana University, Indianapolis, IN (US)

(72) Inventors: Iman Nabiyouni, San Mateo, CA (US); John Shea, Bloomington, IN (US)

(73) Assignee: The Trustees of Indiana University, Indianapolis, IN (US)

(21) Appl. No.: 16/778,354

(22) Filed: Jan. 31, 2020

Related U.S. Application Data
(60) Provisional application No. 62/799,297, filed on Jan. 31, 2019.

(51) Int. Cl. A61B 5/00; A61B 5/103; A43B 3/00

(52) U.S. Cl. CPC A61B 5/6807; A61B 2562/0247; A43B 3/0005; A61B 5/1038

(57) ABSTRACT

A simplified, inexpensive, wearable system, and methods using the system, to determine an individual's postural stability and center of pressure, the determination occurring with enhanced accuracy compared to current systems, and occurring in real-time.

This patent application claims the benefit of, and priority to, U.S. Provisional Patent Application Ser. No. 62/799,297, the disclosure of which is incorporated herein by reference.

Balance can be an informative source of information in daily life. Balance can show the level attention that our brain is devoting to a physical task (e.g. distraction with cellphone or being drunk during walking or standing still). In addition to the mental attention, balance can also be used to represent physical condition. More specifically this gait evaluation can reveal level of fatigue or risk of fall as a safety tool. Also it can be used as an evaluation tool e.g. diagnosing functional deficit or monitoring performance improvement in rehabilitation. Center of pressure (CoP) is one measure of postural stability. A need exists to employ this helpful technique and service commonly. This inventive systems and methods address this need by providing simplified, inexpensive, wearable systems and methods to determine an individual's postural stability and CoP in real-time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A compares mapped centers of pressure (CoP) for a conventional force platform to an inventive system.

FIG. 1B compares x displacements of CoP versus time for a conventional force platform to an inventive system.

FIG. 1C compares y displacements of CoP versus time for a conventional force platform to an inventive system.

FIG. 2 shows an insole according to an embodiment of the invention.

FIG. 3 shows a coordination monitoring sensor according to an embodiment of the invention.

FIG. 4 shows dynamic Center of Pressure (dCOP) with embedded position sensor in walking.

FIG. 5 shows a schematic of an insole with/without an embedded position sensor.

One embodiment of the invention is a wearable system to determine an individual's center of pressure. The system includes an article of footwear including an insole; one or more input sensors for measuring pressure housed in the shoe insole, where the one or more input sensors are configured to produce pressure information proportional to mechanical pressure placed on the one or more input sensors; a communication module housed outside the article of footwear and coupled with the one or more input sensors, where the communications module is configured to receive the pressure information from the one or more input sensors and further configured to transmit the pressure information to a computer; optionally, a motion sensor configured to receive positional information of the article of footwear and transmit the positional information to the computing device; and, a computing device including a second communications transceiver coupled with the communications medium, where the computing device is configured to receive the pressure information from the communications module using the second communications transceiver and to receive the positional information from the motion sensing device and determine center of pressure from the pressure information and the positional information. The one or more input sensors may collect a distributed force data, preferably over an area of about 12 cm². The one or more input sensors may be embedded force detectors. The embedded force detectors may be force sensitive resistors. The communication module may include a first communication transceiver coupled with a communication medium. The communication medium may be a wireless communications medium.

The computing device may determine center of pressure using an algorithm. In some embodiments, the determined center of pressure is local center of pressure and the determined center of pressure may be a global center of pressure. In embodiments, the system may be markerless.

Another aspect of the invention is a method to determine center of pressure of a person by providing an article of footwear worn by the person having an insole portion; providing one or more input sensors for measuring pressure in the insole portion of the article of footwear; reading the one or more input sensors; providing one or more a motion sensors for measure a position of the article of footwear; generating pressure information proportional to the mechanical pressure placed on the one or more input sensors; generating position information for the article of footwear; transmitting the pressure information and the position information; receiving the transmitted pressure information and the transmitted position information; and, calculating center of pressure for the person based on the received pressure data information. The determined center of pressure may be a local center of pressure or a global center of pressure. The input sensors may be all of the same construction or of different construction, as is well known in the art. The location and size of the input sensors may vary depending upon the specific application for which the system is designed.

In embodiments, the method also includes the step of notifying a third party monitoring person if the postural state information indicates a predetermined condition, where the predetermined condition includes a postural state selected from the group consisting of stable postural state, an unstable postural state, and a partially stable postural state. The third party monitoring person is selected from the group consisting of a physician, a physician's assistant, an occupational therapist, a nurse, a physical therapist, a person trainer, or a recreational therapist. In addition, the methods may include the step of calculating center of pressure for a gait of the person over multiple steps.

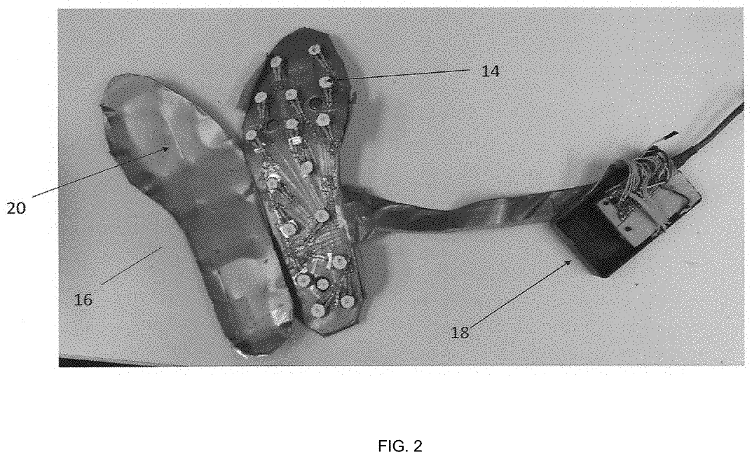

According to one embodiment, FIG. 2 is an exemplary wearable system to determine an individual's postural stability, i.e., the individual's center of pressure. Alternatively, the system may be used to determine postural stability in robots and animals. In the exemplary embodiment, the system comprises an article of footwear (not shown) worn by a person including an insole (16). Exemplary articles of footwear include any footwear that may be configured to house an insole such as dress shoes, open-toe shoes, closed-toe shoes, running shoes, walking shoes, boots, and ski boots. The shoe insole (20) may be manufactured from any suitable insole materials including, without limitation, foam, rubber, plastic, cork, and/or other materials suitable for shoe insole construction. The shoe insole material selection may be determined from factors including durability, flexibility, and/or protection of internal components. The inventive system may be configured for insoles of all shoe sizes and widths, for toddlers, children and adults.

One or more input sensors (14) are housed in the insole (16) for measuring a pressure at different locations underneath one or both of an individual's feet. The one or more input sensors (14) are configured to produce pressure information proportional to mechanical pressure placed on the one or more input sensors. In embodiments of the invention, the one or more input sensors (14) are embedded force detectors. In other embodiments, the embedded force detectors are force sensitive resistors. In further embodiments, the one or more input sensors (14) collect a distributed force data over an area of about 12 cm².

The one or more input sensors (14) are affixed to an underside of an insole using a layout such that the input sensors cover important foot plantar pressure distribution areas such as big toe, metatarsophalangeal joint, arch of foot, and heel. It can be possible to replace all sensors with one special designed sensor that cover plantar areas and can monitor the pressure with its location in each area. In embodiments, an insole coordinate system may be used to identify an actual location of the pressure information on an insole for data interpretation and reproducibility. It may be desirable to have any number of input sensors (14) in each insole, placement of input sensors in a variety of locations, and possibly different numbers of input sensors in each insole. In the case of having the special designed sensor, it is possible to have any size of the sensor which cover some or all plantar areas. In embodiments, the number of input sensors engaged to an insole will range from about 2 input sensors to about 20 input sensors inclusive, e.g., 3, 4 up to 19, 20 input sensors. It will be appreciated by one skilled in the art that sensor number and position may be adapted for various applications that require different dimensionality of center of pressure information.

The inventive wearable system to determine postural stability such as center of pressure further comprises one or more communications modules (18) housed outside the insole and coupled with the one or more input sensors. The sensor information is transmitted from the input sensors (14) to the communication module (18). For example, the input sensors (14) may be pressure sensors, and transmission from the input sensors (14) to the communication module (18) can be via external wires and/or via wires embedded in the insole. The communications module is configured to receive the pressure information from the one or more input sensors (14) and is further configured to transmit the pressure information to an external computer device. In the case of having one special designed sensor that can measure the pressure along with its location, the communication module may also receive the location data from each sensing area. The communications module (18) may transmit the data to the computing device using a communications medium or link. Preferably, the communications link is a wireless link such as a Wi-Fi link. Other communications links such as a Bluetooth link, an infrared link or a USB link are known in the art. It will be appreciated that communications links may comprise any type of wireless link known in the art. Additionally, the communication modules may optionally perform other processing steps on the sensor information before transmitting the sensor information to the computing device (e.g., linearity correction, data transform, etc.).

It will be appreciated that communications modules may comprise one or more integrated circuits and/or discrete components on a printed circuit board, a flexible printed circuit board, or other electronic packaging technology. A power source such as a battery may be attached by any suitable arrangement for providing power to the circuits of the communications module. Communications module may be located inside the insole, in the shoe upper, the shoe sole, and/or another location in the shoe. Transmission of information between input sensors and communications module may be by wires embedded in the insole, shoe sole, and/or shoe upper. It will be appreciated by one skilled in the art that communications module may be split into a number of components, the components being located in any portion of the shoe or the insole and connected by wires or other connecting technology to achieve the functionality of communications module described above.

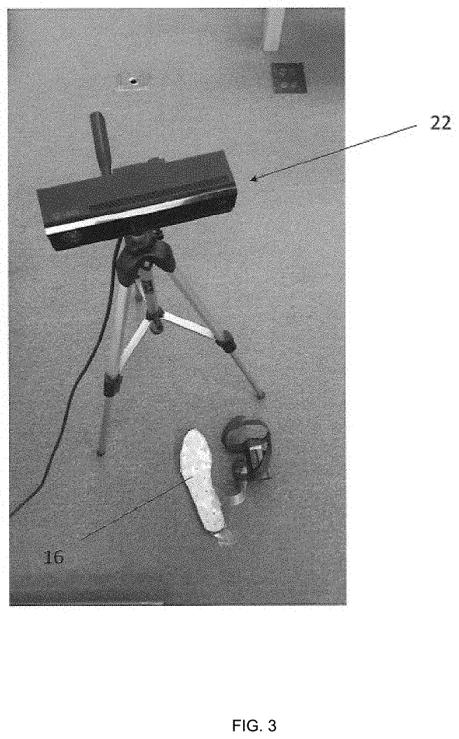

As shown in FIG. 3, the inventive system requires local/global position/distance of each foot/leg from a source. In the case of using global position/distance of each foot/leg, the inventive system further includes a coordination monitoring sensor (22) optionally mounted on a tripod, and configured to provide global positional information for the insole, the article or articles of footwear on the wearer and to transmit the positional information to the computing device. In embodiments, positional information for one or both articles of footwear can be captured and/or analyzed using a motion capture or range camera such as a NOAH™ system, a coordination monitoring sensor or a KINECT™ system. In the case of using local position/distance of each foot/leg, the inventive system further includes a motion sensor that can be placed in the insoles or attached to any other part of each leg. This sensor can provide its relative/local location/distance information from a source which is a device and is placed in any other parts of the body The motion sensor may transmit the data to a computing device using a communications link. The communications link may be any wired or wireless link known in the art.

Computing device contains instructions on a machine readable medium to receive the pressure with/without its local location information from the pressure sensing device and to receive the positional/distance information from the motion sensing device and determine center of pressure from the pressure with/without its local location information and the positional information. In embodiments, the determination of center of pressure utilizes a posterior decoding algorithm, a Bayesian segmentation, a graphical model, a choice-point method, and/or any other type of algorithm. An exemplary algorithm for determining center of pressure is disclosed in Table 2.

In embodiments, the computing device may be associated with the person that is using the inventive system. In alternate embodiments, the computing device can be utilized by a third party (e.g., doctor, physical therapist, personal trainer, etc.) to track the center of pressure of the person to track the progress of a patient or to assess a patient's risk of fall. The medical professional could receive detailed real-time information about the patient's postural stability and make an appropriate medical diagnosis. Further, the medical professional can take corrective action based on the gathered data to fix problems with the patient's postural stability. Also the computing device can be utilized with any one to monitor the mental attention/physical condition of the user. The monitoring person could receive detailed real-time information about the postural stability to make appropriate action.

Embodiments of the invention also include methods to determine postural stability, such as an individual's center of pressure. These methods include the steps of providing an article of footwear worn by the person having an insole portion; providing one or more input sensors for measuring pressure in the insole portion of the article of footwear; reading the one or more input sensors; providing one or more motion sensors for measure a position of the article of footwear; generating pressure with/without location information proportional to the mechanical pressure placed on the one or more input sensors; generating position information for the article of footwear; transmitting the pressure with/without location information and the position information; receiving the transmitted pressure with/without location information and the transmitted position information; and calculating center of pressure information for the person based on the received pressure with/without location information and position information.

In embodiments, the determined center of pressure is a local center of pressure, defined as center of pressure which is calculated relative a local source/coordinate. In other embodiments, the determined center of pressure is global center of pressure, defined as center of pressure which is calculated relative a global source/coordinate. The local center of pressure can be calculated with/without having the position of each foot/leg.

In the case of not having the position of each leg/foot, the center of pressure just can include the single support portion of walking cycle (known as stride). The missing position of walking (which is double support phase of stride) can convey helpful information about the balance as human is more stable in this phase of walking and may relatively increase to make them more stable (e.g. in falling scenarios).

However, using the position of each foot/leg make it possible to calculate local/global center of pressure for the whole stride.

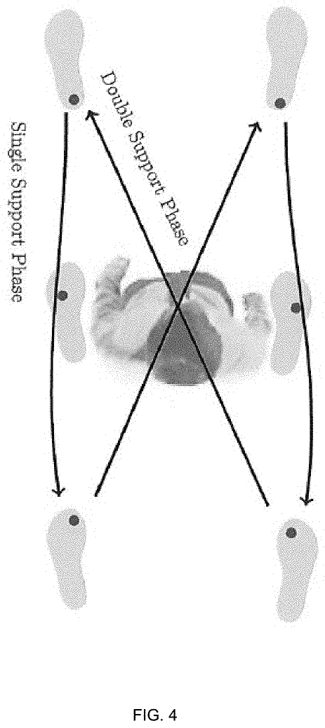

As shown in FIG. 4, black arrows represent dCoP in a stride (i.e., internal arrows for double support phase and external arrows for single support phase of walking). Green dots depict position of dCoP with respect to sole. Embedded position sensor in the insole measures the local position with respect to an origin. The local origin can be a portable computing device (e.g. cellphone).

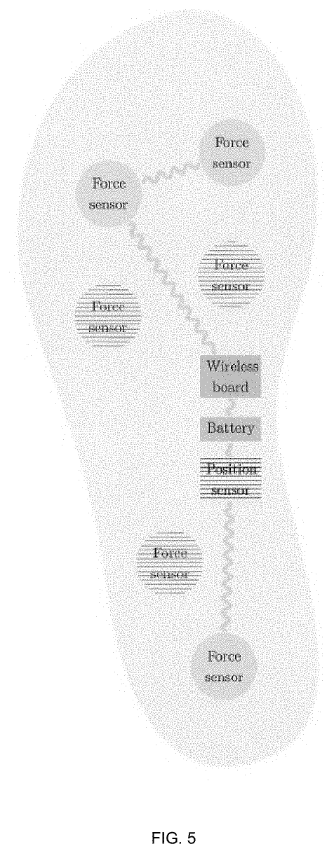

As shown in FIG. 5, red circles represent force sensors. This can be one large sensor (which covers most of the plantar area) or multiple small sensors. Blue box depicts position sensor which measures position in local coordinate system. This sensor can be replaced with a motion tracking system which measures position in global coordinate system (e.g., Microsoft Kinect). Dark green box illustrates a board for transferring force and position data to a measuring device (e.g., cellphone). Powers supply for this circuit is provided with a battery which is shown by a bright green box.

Two healthy subjects (age 18-35) performed two blocks of 20 trials on a force platform (AMTI OR6-7-1000, Advanced Mechanical Technology, 16 Inc., Watertown Mass.) while wearing the inventive insoles. After calibration of the devices, in the first block, the two subjects stood behind the platform and took two steps on the platform starting with right and left foot (5 trials each). A second block of trials included standing on the force platform and taking a step with either of the feet for 5 times (gait initiation). Timing was synchronized by sending a digital signal from the insole software to the force platform recording device. In analyzing the data, the Pearson's correlation coefficient (p) and the concordance correlation coefficients (cc) was calculated which are displayed in Table 1; p=Pearson's correlation coefficient, cc=concordance correlation coefficients. Correlation coefficients have been calculated between CoP displacements of force platform and insoles in x and y direction. In the first column, type of the task and in the second column leading foot is specified.

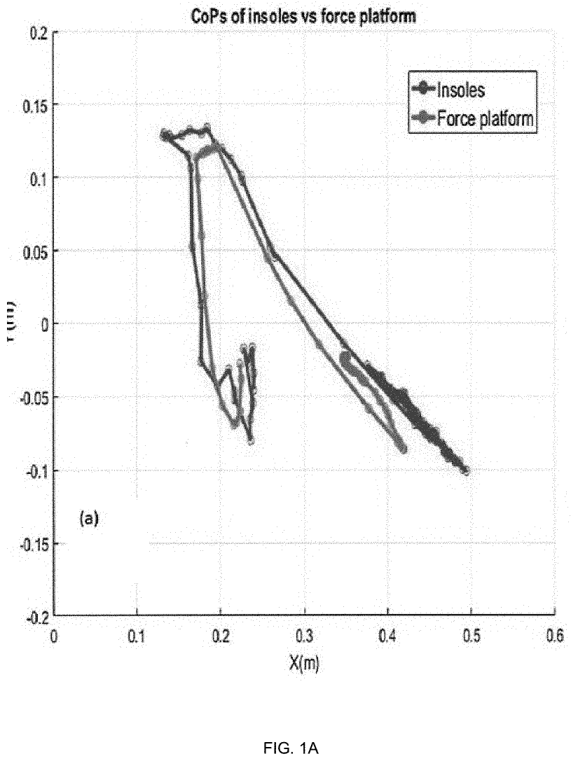

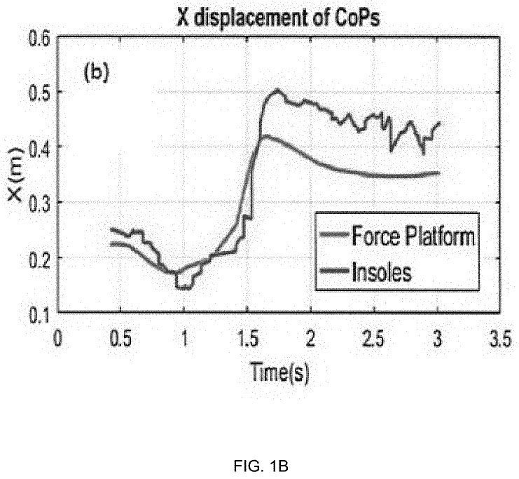

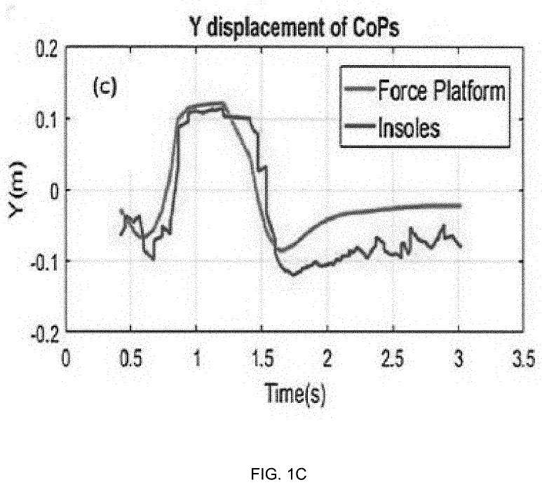

The results show acceptable agreement between the insoles and force platform data, and provide a basis for extending this methodology to all population as in a wearable device. Two-step tasks in x and y direction had the p of 0.97 and 0.98 (in x and y direction) and cc of 0.95 and 0.97. That of gait initiation trials were 0.96, 0.97, 0.95 and 0.96 respectively which confirm an excellent correlation in range of 0.75-1. An example of mapped CoPs for a gait initiation trial is shown in FIGS. 1A-1C.

TABLE 1
Correlation Coefficients of CoP data

Correlation Coefficient Data

| Task | Foot | p-x | p-y | cc-x | cc-y |
|---|---|---|---|---|---|
| Gait | Left | 0.98 | 0.89 | 0.92 | 0.83 |
| Initiation | Right | 0.96 | 0.92 | 0.86 | 0.86 |
| Two-step | Left | 0.98 | 0.98 | 0.95 | 0.96 |
| | Right | 0.95 | 0.96 | 0.95 | 0.96 |

TABLE 2
CoP algorithm
Algorithm 1: Calculating global CoP

| | while record_data is True |
|---|---|
| 1 | for left and right foot |
| 2 | // get position and measured force of each sensor |
| 3 | F_s, P_s = get_Sensors_Data(reading_size) F_cs = |
| | remove_noise(F_s) |
| 4 | // calculate local CoP of each foot separately |
| 5 | CoP_lx, CoP_ly= cal_local_CoP(F_cs, P_s) |
| 6 | // get position of heels and toes from Kinect |
| 7 | X, Y= get_feet_pos( ) |
| 8 | // rotate and align the local CoP with each foot angle |
| 9 | CoP_lxA, CoP_lyA= rot_to_global(CoP_lx, CoP_ly, X, Y) |
| 10 | End |
| 11 | // calculate global CoP for both feet with subscripts l and r |
| 12 | CoP_gx, CoP_gy= cal_global_CoP(Xₗ; Yₗ;Xᵣ; Yᵣ;CoP lxAₗ; |
| | CoP lyAₗ;CoP lxAᵣ;CoP lyAᵣ; F sₗ; F_sᵣ) |
| 13 | // Draw the result on the screen |
| 14 | Draw(CoP_gx, CoP_gy) end |
| 15 | End |

Algorithm 2 - Procedure remove_noise(F_s)

| | |
|---|---|
| 1 | remove_noise(F_s) |
| 2 | X = F_s |
| 3 | epsilon = small number |
| 4 | Initialize \vec{β} |
| 5 | while error > epsilon // finding β₁ to βₙ₊₁ that minimize the error |
| 6 | \vec{y} = X\vec{β} + \vec{ϵ} |
| 7 | \vec{β} = (X^{T}X)⁻¹X^{T}\vec{y} |
| 8 | error = ∥\vec{y} - X\vec{β}∥ |
| 9 | End |
| 10 | return β₁F_sⁿ + β₂F_sⁿ⁻¹+... + βₙF_s+ βₙ₊₁ |

Algorithm 3 − Procedure cal_local_CoP(F_cs, P_s)

| | |
|---|---|
| 1 | cal_local_CoP(F_cs, P_s) |
| 2 | return F_cs · P_s/∥F_cs∥ |

Algorithm 4 − Procedure rot_to_global(CoP_lx, CoP_ly, X, Y)

| | |
|---|---|
| 1 | rot_to_global(CoP_lx, CoP_ly, X, Y) |
| 2 | ang_foot = arctan((y₂ − y₁)/(x₂-x₁)) |
| 3 | ${rot}_{m} at = [ \begin{matrix}cos ang\_foot & sin ang\_foot \\ - sin ang\_foot & cos ang\_foot\end{matrix} ]$ |
| 4 | CoP_lyA,Cop_lxA = rotₘat · CoP_{ly}, Copₗₓ |
| 5 | return CoP_lyA, CoP_lxA |

Algorithm 5 − Procedure cal_global_CoP(Xₗ, Yₗ, ...)

| | |
|---|---|
| 1 | cal_global_CoP(Xₗ, Yₗ, Xᵣ, Yᵣ, CoP_lxAₗ, CoP_lyAₗ, CoP_lxAᵣ, |
| | CoP_lyAᵣ, F_sₗ, F_sᵣ) |
| 2 | X_gₗ = Xₗ + CoP_lxaₗ |
| 3 | X_gᵣ = Xᵣ + CoP_lxaᵣ |
| 4 | Y_gₗ = Yₗ + CoP_lyaₗ |
| 5 | Y_gᵣ = Yᵣ + CoP_lyaᵣ |
| 6 | ${CoP}_{g} x = \frac{{X\_g}_{l} \cdot {F\_sx}_{l} + {X\_g}_{r} \cdot {F\_sx}_{r}}{{F}_{s} {x}_{l} + {F\_sx}_{r}}$ |
| 7 | ${CoP}_{g} y = \frac{{Y\_g}_{l} \cdot {F\_sy}_{l} + {Y\_g}_{r} \cdot {F\_sy}_{r}}{{F}_{s} {y}_{l} + {F\_sy}_{r}}$ |
| 8 | return CoP_{g}x, CoP_{g}y |

The algorithm in Table 2 first detects a local CoP of each foot (with respect to a local source in the plantar areas) and then uses the location of each foot during walking to determine a global CoP. In the algorithm of Table 2, by knowing the position of each sensor based on the design of the insole, sensor values are read as wireless data from an open socket. A noise reduction technique is then employed to remove contamination from the signal. This process is performed over iterations and finds the best fitted curve that can represent the data. In the next step, local CoP, with regards to each foot, is calculated, which is referred to as “CoP_I”. This local CoP determines CoP for each foot separately. This value is limited to the area of each sole and usually changes from heel to toe. Most of the commercially available insoles now used to measure postural stability are only able to measure this local CoP for each foot. Advantageously, the inventive system measures global CoP. Global CoP is more informative since it also includes the local CoP inside. Global CoP is a more reliable measure of balance due to its sensitivity to changes in body posture. To measure global CoP, a value for the position of each as provided by the motion sensor of the inventive system is used. Once foot position is determined, a local position of each foot can be mapped to the global coordinate system (Kinect coordinate system). This mapping include rotation and transformation of local CoP. The local CoP of each insole is first transformed to the global coordinate system and then global CoP is calculated in an X and a Y direction. The local CoP is also referred to as in-shoe CoP. Other prior insoles are just a transformation of local CoPs to global coordinate without considering their relative forces. Because of this lack of consideration, prior methods cannot provide CoP in the double support phase of walking which is an important phase for keeping balance. The result of these methods is an intermittent CoP.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

### REFERENCES

Fleiss, (1986). Design and analysis of clinical experiments. New York, USA: John Wiley & Sons.
Chesnin et al. Comparison of an in-shoe pressure measurement device to a force plate: concurrent validity of center of pressure measurements. Gait & posture, 12(2):128-133, 2000.
Fradet et al. Spatial synchronization of an insole pressure distribution system with a 3d motion analysis system for center of pressure measurements. Medical & biological engineering & computing, 47(1):85-92, 2009.
Hase and Stein. Analysis of rapid stopping during human walking. Journal of neurophysiology, 80(1):255-261, 1998.
Oren Tirosh and W A Sparrow. Gait termination in young and older adults: effects of stopping stimulus probability and stimulus delay. Gait & posture, 19(3):243-251, 2004.
Weaver et al. A direct method for mapping the center of pressure measured by an insole pressure sensor system to the shoe's local coordinate system. Journal of Biomechanical Engineering, 138(6):061007, 2016.
O'Loughlin, J. L., Robitaille, Y., Boivin, J-F., Suissa, S. Incidence of an risk factors for falls and injurious falls among the community-dwelling elderly. American Journal of Epidemiology, 137 (3): 342-354, 1993.
Boulgarides, L. K., McGinty, S. M., Willett, J. A., Barnes, C. W. Use of clinical and impairment-based tests to predict falls by community-dwelling older adults. Physical Therapy, 83 (4): 328-339, 1993.

1. A wearable system to determine an individual's center of pressure, the system comprising
an article of footwear including an insole;
one or more input sensors for measuring pressure housed in the shoe insole, where the one or more input sensors are configured to produce pressure information proportional to mechanical pressure placed on the one or more input sensors;
a communication module housed outside the article of footwear and coupled with the one or more input sensors, where the communications module is configured to receive the pressure information from the one or more input sensors and further configured to transmit the pressure information to a computer;
optionally, a motion sensor configured to receive positional information of the article of footwear and transmit the positional information to the computing device; and,
a computing device including a second communications transceiver coupled with the communications medium, where the computing device is configured to receive the pressure information from the communications module using the second communications transceiver and to receive the positional information from the motion sensing device and determine center of pressure from the pressure information and the positional information.

2. The system of claim 1 where the one or more input sensors collect a distributed force data over an area of about 12 cm².

3. The system of claim 1 where the one or more input sensors are embedded force detectors.

4. The system of claim 3 where the embedded force detectors are force sensitive resistors.

5. The system of claim 1 where the communication module includes a first communication transceiver coupled with a communication medium.

6. The system of claim 5 where the communication medium comprises a wireless communications medium.

7. The system of claim 1 where the computing device determines center of pressure using an algorithm.

8. The system of claim 1 where the determined center of pressure is local center of pressure.

9. The system of claim 1 where the determined center of pressure is global center of pressure.

10. The system of claim 1 where the system is markerless.

11. A method to determine center of pressure of a person, the method comprising
providing an article of footwear worn by the person having an insole portion;
providing one or more input sensors for measuring pressure in the insole portion of the article of footwear;
reading the one or more input sensors;
providing one or more a motion sensors for measure a position of the article of footwear;
generating pressure information proportional to the mechanical pressure placed on the one or more input sensors;
generating position information for the article of footwear;
transmitting the pressure information and the position information;
receiving the transmitted pressure information and the transmitted position information; and,
calculating center of pressure for the person based on the received pressure data information.

12. The method of claim 11 where the determined center of pressure is local center of pressure.

13. The method of claim 11 where the determined center of pressure is global center of pressure.

14. The method of claim 11 further comprising notifying a third party monitoring person if the postural state information indicates a predetermined condition, where the predetermined condition includes a postural state selected from the group consisting of stable postural state, an unstable postural state, and a partially stable postural state.

15. The method of claim 14 where the third party monitoring person is selected from the group consisting of a physician, a physician's assistant, an occupational therapist, a nurse, a physical therapist, a person trainer, or a recreational therapist.

16. The method of claim 11 further comprising calculating center of pressure for a gait of the person over multiple steps.
